(19) United States
(12) Patent Application Publication
(10) Pub. No.: US 2004/0052787 A1
(43) Pub. Date: Mar. 18, 2004

(54) Methods for treating disorders of the female reproductive system

(76) Inventors: Anne Elizabeth King, Edinburgh (GB); Hilary Octavia Dawn Critchley, Edinburgh (GB); Rodney William Kelly, Edinburgh (GB); Richard P. Phipps, Pittsford, NY (US)

Correspondence Address: Michael L Goldman, Nixon Peabody, Clinton Square, PO Box 31051, Rochester, NY, 14603, US

(21) Appl. No.: 10/399,166

(22) PCT Filed: Oct. 15, 2001
(86) PCT No.: PCT/GB01/04566

(30) Foreign Application Priority Data
Oct. 13, 2000 (GB) 0025132.2

(51) Int. Cl. A61K 39/395; C07K 16/28

(52) U.S. Cl. 424/144.1; 530/388.22

(57) ABSTRACT

A method of treating a disorder of the female reproductive system in an individual the method comprising modulating the CD40-CD40L system in the said reproductive system. Typically, an agent is used which disrupts the CD40-CD40L system. Typically, the disorder of the female reproductive system is a menstrual dysfunction, preterm labour or endometriosis. A screening method for predisposition of an individual to a disorder of the female reproductive system, the method comprising obtaining a sample form the individual which contains CD40 and/or CD40L nucleic acid or protein and determining whether CD40 and/or CD40L nucleic acid or protein is present in an amount and/or concentration, or in a form, indicative of such predisposition.

The present invention relates to methods for treating disorders of the female reproductive system.

It is known that disorders can occur in tissues and organs of the female reproductive system. Examples of these disorders include menstrual dysfunction, pre-term labour and endometriosis.

The present invention is based on the finding that disorders such as menstrual dysfunction and pre-term labour are associated with an increase in levels of certain inflammatory moieties, and that this may be mediated by the CD40-CD40L system which, until the present work, had not been detected in endometrial tissue.

In particular, the present invention relates to treatment of disorders in tissues and organs of the female reproductive system, such as menstrual dysfunction and pre-term labour, using an agent that can modulate, such as reduce or eliminate or alleviate, the adverse effects of one or more of those inflammatory moieties. Typically, the agent is one which modulates the CD40-CD40L system.

The present invention also relates to agents, and compositions (especially pharmaceutical compositions) comprising same, that can modulate, such as reduce or eliminate or alleviate, the adverse effects of one or more of those inflammatory moieties. Typically, the agent is one which modulates the CD40-CD40L system.

As indicated above, the present invention is based on the finding that it may be possible to treat some disorders of the female reproductive system by modulating the adverse effects of one or more inflammatory moieties, typically by modulating the CD40-CD40L system.

Here, the term “inflammatory moieties” means moieties that are capable of causing, directly or indirectly, an inflammatory effect or a number of inflammatory effects, that cause, directly or indirectly, disorders of the female reproductive system—such as menstrual dysfunction, pre-term labour and endometriosis.

Typical inflammatory moieties are inflammatory cytokines or inflammatory chemokines.

As indicated, typical inflammatory moieties are inflammatory cytokines—viz cytokines that are capable of causing, directly or indirectly, an inflammatory effect or a number of inflammatory effects, that cause, directly or indirectly, disorders of the female reproductive system—such as menstrual dysfunction, pre-term labour and endometriosis.

Cytokines are typically proteins that are released by mammalian cells and act on other cells through specific receptors. They elicit from the target cell a variety of responses depending on the cytokine and target cell. Cytokine actions include control of cell proliferation and differentiation, regulation of immune responses, hemopoiesis, and inflammatory responses.

Examples of inflammatory cytokines are certain interleukins, in particular. IL6.

As indicated, typical inflammatory moieties are inflammatory chemokines—viz chemokines that are capable of causing, directly or indirectly, an inflammatory effect or a number of inflammatory effects, that cause, directly or indirectly, disorders of the female reproductive system—such as menstrual dysfunction, pre-term labour and endometriosis.

The term “chemokine”, is a contraction of “chemotactic cytokines”. The chemokines comprise a large family of proteins which have in common important structural features and which have the ability to attract leukocytes. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory and infectious diseases, agents which are active in modulating, preferably antagonizing, the activity of chemokines and their receptors, are useful in the therapeutic treatment of such inflammatory and infectious diseases.

Examples of inflammatory chemokines are certain interleukins, in particular IL8, and monocyte chemoattractants, such as monocyte chemoattractant peptides, more in particular monocyte chemoattractant peptide 1 (MCP-1).

The cytokines and chemokines discussed are well known in the art; for example, see Ibelgaufts, H. (1995) Dictionary of Cytokines, Weinheim, VCH.

An important finding of the present invention is that if one targets the CD40-CD40L system of fibroblasts or myofibroblasts in tissues and organs of the female reproductive system then it is possible to modulate, such as reduce or eliminate or alleviate, the adverse effects of one or more of the inflammatory moieties.

As is well known, a fibroblast is a flattened, irregular, branched, motile cell found throughout vertebrate connective tissue. Fibroblasts form, secrete and maintain the extracellular collagen and mucopolysaccharide of this tissue. Similar cells occur in many invertebrates. Myofibroblasts are cells with fibroblast character and smooth muscle character characterised by smooth muscle actin expression.

In particular we have found that by targeting the CD40-CD40L system of fibroblasts in tissues and organs of the endometrium and/or myometrium and/or cervix and/or decidua and/or trophoblast is possible to modulate, such as to reduce or eliminate or alleviate, the adverse effects of one or more of the inflammatory moieties. The endometrium may be perimenstrual endometrium, proliferative endometrium or secretory endometrium, and either eutopic or ectopic.

CD40-CD40L system is made up of a cell surface glycoprotein (CD40) found in fibroblasts and the ligand for CD40 (ie CD40L). The system is described in Ibelgauft, H. (1995) Dictionary of Cytokines, Weinheim, VCH.

Background teachings on CD40 and CD40L have also been presented by Victor A. McKusick et al on http://www.ncbi.nlm.nih.gov/Omim. The following information concerning CD40 and CD40L has been extracted from that source.

CD40, a 48-kD glycoprotein, is expressed on the surface of all mature B cells, most mature B-cell malignancies, and some early B-cell acute lymphocytic leukemias, but is not expressed on plasma cells (Clark, 1990). Stamenkovic et al (1989) isolated a cDNA encoding CD40 and demonstrated by the predicted sequence of the protein that CD40 is related to human nerve growth factor receptor. It is also closely related to the receptor for TNF-alpha and to CD27. These homologies imply that the ligand for CD40 may be a soluble factor and that CD40 is a member of the cytokine receptor family. CD40 is a phosphoprotein and is capable of expression as a homodimer.

Using chromosomal in situ hybridization, Lafage-Pochitaloff et al (1994) localized the CD40 gene to 20q12-ql3.2. This localization correlated well with the mapping of the murine CD40 gene to the distal region of chromosome 2 which shows rather extensive homology of synteny to human 20q11-q13.

By analysis of lymphoblastoid cell lines carrying. 20q deletions, Asimakopoulos et al (1996) placed CD40 within a 19- to 21-cM interval that was almost coincidental with the common deleted region defined by previous analysis of samples from patients with myeloid malignancies. CD40-CD40-ligand (CD154) interactions play a critical role in immune activation. Using replication defective adenovirus encoding mouse CD154 (Ad-CD154), Kato et al (1998) modified human chronic lymphocytic leukemia B cells to express a functional ligand for CD40. This not only induced expression of immune accessory molecules on the infected cell, but also allowed it to transactivate noninfected bystander leukemia B cells. Also, factors that impair the antigen-presenting capacity of leukemia B cells were downmodulated. Kato et al (1998) suggested that Ad-CD154 can induce a host antileukemia response that may have therapeutic potential.

Increasing evidence supports the involvement of inflammation and immunity in atherogenesis. Cells in human atherosclerotic lesions express the immune mediator CD40 and its ligand CD40L, deficiency of which is responsible for X-linked immunodeficiency with hyper-IgM. The interaction of CD40 with CD40L figures prominently in both humoral and cell-mediated immune responses. CD40L-positive T cells accumulate in atheroma, and, by virtue of their early appearance, persistence, and localization at sites of lesion growth and complication, activated T cells may co-ordinate important aspects of atherogenesis. Interruption of CD40L-CD40 signalling by administration of an anti CD40L antibody was found to limit experimental autoimmune diseases such as collagen induced arthritis, lupus nephritis, acute or chronic graft-versus-host disease, multiple sclerosis, and thyroiditis. Ligation of CD40 on atheroma-associated cells in vitro activates functions related to atherogenesis. Mach et al (1998) determined whether interruption of CD40 signalling influences atherogenesis in vivo in hyperlipidemic mice. Treatment with antibody against mouse CD40L limited atherosclerosis in mice lacking the receptor for low density lipoprotein that had been fed a high-cholesterol diet for 12 weeks. The antibody reduced the size of aortic atherosclerotic lesions by 59% and their lipid content by 79%. Furthermore, atheroma of mice treated with anti-CD40L antibody contained significantly fewer macrophages (64%) and T lymphocytes (70%), and exhibited decreased expression of vascular cell adhesion molecule-1 (VCAM-1). These data supported the involvement of inflammatory pathways in atherosclerosis and indicated a role of CD40 signalling during atherogenesis in hyperlipidemic mice.

Alzheimer disease has a substantial inflammatory component, and activated microglia may play a central role in neuronal degeneration. Tan et al (1999) demonstrated that the CD40 expression was increased on cultured microglia treated with freshly solubilized amyloid-beta and on microglia from a transgenic murine model of Alzheimer disease (Tg APPsw).

Increased TNF-alpha production and induction of neuronal injury occurred when amyloid-beta-stimulated microglia were treated with CD40 ligand. Microglia from Tg APPsw mice deficient for CD40 ligand had less activation, suggesting that the CD40-CD40 ligand interaction is necessary for amyloid-beta-induced microglial activation. In addition, abnormal tau phosphorylation was reduced in Tg APPsw animals deficient for CD40 ligand, suggesting that the CD40-CD40 ligand interaction is an early event in Alzheimer disease pathogenesis.

A first aspect of the present invention provides a method of treating a disorder of the female reproductive system in an individual the method comprising modulating the CD40-CD40L system in the said reproductive system.

By “CD40-CD40L system” we mean CD40 and CD40L (also known as CD154) and their interaction. As noted above, the CD40-CD40L system has been investigated in mice and humans but it is only with the present work that it has surprisingly been found in the normal human reproductive system, in particular in a perivascular location in the normal endometrium and myometrium.

By “disorder of the female reproductive system” we include any such disorder, but it is particularly preferred if the disorder is a benign condition and is not a reproductive cancer. Additionally, it is preferred if the disorder is one involving vasculature. Typical disorders of the female reproductive system which can be treated using the method of the invention include menstrual dysfunction such as menorrhagia and dysmenorrhoea, pre-term labour, endometriosis and uterine fibroids.

Common endometrial pathologies such as dysmenorrhoea and menorrhagia are both caused by dysfunctional events in the cells immediately surrounding the blood vessels. Prostaglandins have been implicated in both of these disorders. PGE is both vasoactive allowing oedema and cellular egress from vessels and is hyperalgesic. Progesterone appears to keep prostaglandin levels low in these cells that surround the vessels (perivascular cells) (Cheng, Kelly et al (1993) J. Clin. Endocrinol. Metab. 77 (qu1), 873-877). The discovery of CD40 expression in these perivascular cells shows that the CD40-CD40L system can control prostaglandin levels in these loci and since it is known that CD40 downstream signalling proceeds usually through the NFκB pathway which is progesterone sensitive, the CD40-CD40L system in turn will be modulated by progesterone and sensitised by falling progesterone levels that occur prior to menstruation. The key distribution of CD40 in the perivascular cells allows an unexpected opportunity to treat reproductive disorders, particularly a subset that are the result of over stimulation or incomplete control of the cells associated with the blood vessels of the uterus.

For dysmennorhoea and menorrhagia, administration of the agent is preferably around the time of menstruation. For preterm labour the agent is typically administered between 16 and 30 weeks of gestation.

Modulation of the CD40-CD40L system can be achieved by any suitable means as is discussed in more detail below. Although in the treatment of some disorders it may be beneficial to enhance the interaction between CD40 and CD40L, such as in clearance of endometrial cells and induction of menstruation, (and therefore such enhancement could be used as a contraceptive, possibly in combination with an anti-gestagen), it is typically preferred if the CD40-CD40L interaction is disrupted. Disruption of the CD40-CD40L system is useful in the treatment of menorrhagia, dysmenorrhoea, endometriosis and pre-term labour. It is believed to be particularly useful to alleviate pain associated with endometriosis. Agents which disrupt the CD40-CD40L system are discussed in more detail below.

Thus, a preferred aspect of the invention provides a method of treating a disorder of the female reproductive system in an individual the method comprising disrupting the CD40-CD40L system in the said reproductive system.

In order to achieve the desired modulation (such as disruption) of the CD40-CD40L system, it is preferred if the individual is administered an agent which modulates (such as disrupts) the CD40-CD40L system. The agent which modulates (such as disrupts) the CD40-CD40L system may act directly, for example by interacting directly with one or both components, or it may act indirectly, for example by modulating the expression of one or both components. Typically, the agent is one which targets CD40 or CD40L directly. It is particularly preferred if the agent targets CD40L and prevents it interacting with CD40.

The agent is typically a biologic by which we include, for example, antibodies, variants of CD40 or CD40L, antisense nucleic acid molecules and the like.

Disruption of the CD40-CD40L system using biologics has proven successful in other situations such as transplant rejection (Kirk et al (1999) Nature Medicine 5, 686-693), pulmonary injury (Adawi et al (1998) Am. J. Pathol. 152, 651-657; Adawi et al (1998) Clin. Immunol. Immunopathol. 89, 222-230), and autoimmune disease (Kalled et al (1998) J. Immunol 160, 2158-2165), all of which are incorporated herein by reference. These studies make use of various anti-CD40L monoclonal antibodies which are specifically incorporated into the description by reference.

Suitable agents are described below. It is preferred if the agent is a CD40L antagonist. Suitable CD40L antagonists include antibodies reactive with CD40L, variants of CD40L (typically soluble) which bind to CD40 but which do not activate it, antibodies which bind CD40, prevent CD40L is binding but do not activate CD40 and soluble CD40 which is able to bind CD40L.

A further aspect of the invention provides use of an agent which modulates the CD40-CD40L system in the manufacture of a medicament for treating a disorder of the female reproductive system. Typically, as discussed above, the agent is one which disrupts the CD40-CD40L system.

A still further aspect of the invention provides a therapeutic system for treating a disorder of the female reproductive system the therapeutic system comprising an agent which modulates the CD40-CD40L system in the said reproductive system. Typically, the agent is one which disrupts the CD40-CD40L system.

The therapeutic system may usefully contain a further therapeutic agent. For example, it may be useful to combine treatment with the agent which modulates (such as disrupts) CD40-CD40L system, with treatment with the oral contraceptive pill (combined and progestogen only), progestogens and antigestagens.

Further aspects of the invention include an agent which modulates the CD40-CD40L system for use in treating a disorder of the female reproductive system in an individual and use of an agent which modulates the CD40-CD40L system for treating a disorder of the female reproductive system in an individual. Typically, the agent is one which disrupts the CD40-CD40L system.

A further aspect of the invention provides a pharmaceutical preparation adapted for delivery to the female reproductive system comprising an agent which modulates the CD40-CD40L system. Suitable adaptations for delivery to the female reproductive system are described below.

It is to be appreciated that all references herein to treatment or treating include one or more of curative, palliative and prophylactic treatment.

Preferably, the term treatment includes at least curative treatment and/or palliative treatment.

The therapy (treatment) may be on humans or animals. Preferably, the methods of the invention are used to treat humans.

The therapy may be for treating conditions associated with disorders in female reproductive tissues or organs.

As used herein, the term “agent” may be a single entity or it may be a combination of agents.

The agent may be an antibody. Suitable antibodies are those reactive to CD40L. Typically, these antibodies neutralise the activity of CD40L, for example by preventing its binding to CD40. Antibodies to CD40L which may be suitable for use in the practice of the invention are those described above. In addition, antibodies to CD40L which may be suitable for use in the invention (and which may act as interrupters of the CD40-CD40L interaction) are described in WO 01/30386, WO 00/43032, U.S. Pat. No. 5,961,974, WO 99/45958 and WO 99/38525, all of which are incorporated herein by reference.

Other suitable antibodies are those that bind CD40 but which do not activate it. Typically, these antibodies are ones which are unable to cross-link CD40 (which is one means of activating CD40). Thus, suitable antibodies are ones which are reactive against CD40 but which are univalent (eg Fab fragments, single chain Fv fragments and the like). Antibodies to CD40 are described in WO 99/42075, U.S. Pat. No. 5,674,492 and WO 95/09653, all of which are incorporated herein by reference.

The “antibody” as used herein includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies, for example as described in U.S. Pat. No. 239,400. Neutralizing antibodies, ie, those which inhibit biological activity of the substance polypeptides, are especially preferred for diagnostics and therapeutics.

Antibodies may be produced by standard techniques, such as by immunisation with the substance of the invention or by using a phage display library.

If polyclonal antibodies are desired, a selected mammal (eg, mouse, rabbit, goat, horse, etc) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identified agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against particular epitopes can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; ie, for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256: 495-497), the human B-cell hybridoma technique (Kosbor et al (1983) Immunol Today 4: 72; Cote et al (1983) Proc Natl Acad Sci 80: 2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81: 6851-6855; Neuberger et al (1984) Nature 312: 604-608; Takeda et al (1985) Nature 314: 452-454). Alternatively, techniques described for the production of single chain antibodies (U.S. Pat. No. 4,946,779) can be adapted to produce the substance specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349: 293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256: 1275-1281).

The agent may be a variant or homologue or derivative of CD40L which binds to CD40 but does not activate it (ie a variant which is a CD40L antagonist). Typically, the CD40L variant or homologue or derivative is soluble. Soluble CD40L is described in WO 99/3213S and WO 93/08207 incorporated herein by reference.

The agent may be soluble CD40 which is able to bind CD40L. Soluble CD40 is described in EP 595 659, incorporated herein by reference.

The CD40 amino acid sequence is given in GenBank Accession No X60592, where it is called B-lymphocyte activation molecule (see also Stamenkovic et al (1989) EMBO J. 8, 1403-1410).

Thus, the agent may be an amino acid sequence. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

The agent may typically be a variant, homologue or derivative of CD40L.

The CD40L amino acid sequence is given in GenBank Accession No Z15017 (and SwissProt Accession No P29965) where it is called gp39 (see also Hollenbaugh (1992) EMBO J. 11, 4313-4321).

Here, the term “homologue” means an entity having a certain homology with the subject amino acid sequence (ie CD40L). Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc as the subject amino acid sequence. Although homology can also be considered in terms of similarity (ie amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Calculation of maximum % homology therefore firstly requires the production of an optimal alignment, taking into consideration gap penalties. A suitable computer program for carrying out such an alignment is the GCG Wisconsin Bestfit package (University of Wisconsin, USA; Devereux et al, 1984, Nucleic Acids Research 12: 387). Examples of other software than can perform sequence comparisons include, but are not limited to, the BLAST package (see Ausubel et al, 1999 ibid—Chapter 18), FASTA (Atschul et al, 1990, J. Mol. Biol. 403-410) and the GENEWORKS suite of comparison tools. Both BLAST and FASTA are available for offline and online searching (see Ausubel et al, 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and NCBI.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur ie like-for-like substitution such as basic for basic, acidic for acidic, polar for polar etc. Non-homologous substitution may also occur ie from one class of residue to another or alternatively involving the inclusion of unnatural amino acids such as ornitine (hereinafter referred to as Z), diaminobutyric acid omithine (hereinafter referred to as B), norleucine omithine (hereinafter referred to as O), pyriylalanine, thienylalanine, naphthylalanine and phenylglycine. Replacements may also be made by unnatural amino acids include; alpha* and alpha-disubstituited* amino acids, N-alkyl amino acids*, lactic acid*, halide derivatives of natural amino acids such as trifluorotyrosine*, p-Cl-phenylalanine*, p-Br-phenylalanine*, p-1-phenylalanine*, L-allyl-glycine*, B-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al, PNAS (1992) 89(20), 9367-9371 and Horwell D C, Trends Biotechnol. (1995) 13(4), 132-134.

Other suitable interrupters of the CD40-CD40L system are described in WO 98/58669, WO 98/58672, WO 98/52606, WO 98/01145, WO 97/26000 and WO 95/17202, as well as the above-mentioned patent applications and patents, all of which are incorporated herein by reference.

Other agents which modulate the CD40-CD40L system, for example by disrupting the CD40-CD40L system, may be designed by reference to the crystal structure of CD40L as disclosed in WO 97/00895, incorporated herein by reference, or may be selected in suitable screens.

The agent may be one which modulates the CD40-CD40L system indirectly, for example by modulating expression of either CD40 or CD40L.

Thus, the agent may be an antisense sequence. Antisense sequences which are agents for use in the invention are capable of hybridising to CD40 or CD40L nucleotide sequence. The term “antisense sequence” includes antisense oligonucleotides, such as those between 10 and 30 nucleotides in length, as well as longer sequences. Antisense sequences can be designed by the person skilled in the art by reference to the nucleotide sequences encoding CD40 and CD40L (see GenBank Accession Nos X60592 and Z15017, respectively).

The term “hybridization” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hydridising to the nucleotide sequences presented herein.

Preferably, the term “variant” encompasses sequences that are complementary to sequences that are capable of hydridising under stringent conditions (eg 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hydridising under high stringent conditions (eg 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (eg 50° C. and 0.2×SSC).

In a more preferred aspect, the present invention covers nucleotide sequences that can hybridise to the nucleotide sequence of the present invention, or the complement thereof, under high stringent conditions (eg 65° C. and 0.1×SSC).

In some applications, the polynucleotide for use in the present invention is operably linked to a regulatory sequence which is capable of providing for the expression of the polynucleotide, such as by the chosen host cell. By way of example, the present invention covers a vector comprising the polynucleotide of the present invention operably linked to such a regulatory sequence, ie the vector is an expression vector.

The term “operably linked” refers to a juxtaposition wherein the components described are in a relationship permitting them to function in their intended manner eg to produce an antisense polynucleotide. A regulatory sequence “operably linked” to an antisense sequence is ligated in such a way that expression of the antisense sequence is achieved under condition compatible with the control sequences.

The term “regulatory sequences” includes promoters and enhancers and other expression regulation signals.

The term “promoter” is used in the normal sense of the art, eg an RNA polymerase binding site.

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (eg recombinant DNA).

For some applications, preferably, the nucleotide sequence is cDNA.

For some applications, preferably, the nucleotide sequence may be the same as the naturally occurring form.

Although in general the techniques mentioned herein are well known in the art, reference may be made in particular to Sambrook et al, Molecular Cloning, A Laboratory Manual (1989) and Ausubel et al, Short Protocols in Molecular Biology (1999) 4^{th} Ed, John Wiley & Sons, Inc. PCR is described in U.S. Pat. No. 4,683,195, U.S. Pat. No. 4,800,195 and U.S. Pat. No. 4,965,188.

Many of the agents for use in the method of the invention, such as the CD40 and CD40L variants, are peptides. Synthesis of peptide agents can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent.

Alternatively, the coding sequence of a peptide agent (or variants, homologues, derivatives, fragments or mimetics thereof) may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

Examples of suitable expression hosts for expressing the peptide agents for use in the invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillis niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus lichenizformis, Bacillus amyloliquefaciens, Kluyveromyces lactis and Saccharomyces cerevisiae.

The use of suitable host cells—such as yeast, fungal and plant host cells—may provide for post-translational modifications (eg myristoylation, glycosylation, truncation, lapidation and tyrosine, serine or threonine phosphorylation) as may be needed to confer optimal biological activity on recombinant expression products, such as CD40 and CD40L variants, for use in the present invention.

The agent may be an organic compound or other chemical. The agent includes, but is not limited to, a compound which may be obtainable from or produced by any suitable source, whether natural or not. The agent can be an amino acid sequence (ie a peptide or polypeptide) or a chemical derivative thereof, or a combination thereof. The agent may even be a nucleotide sequence—which may be a sense sequence or an anti-sense sequence.

The agent may act as an inhibitor and/or an antagonist.

The agent may be designed or obtained from a library of compounds which may comprise peptides, as well as other compounds, such as small organic molecules, such as lead compounds.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

It is contemplated that in future further compounds which are able to modulate the CD40-CD40L system will be discovered, such as low molecular weight organic molecules. These are considered to be within the scope of the present invention.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

The agent for use in the present invention may even be capable of displaying other therapeutic properties.

The agent may be used in combination with one or more other pharmaceutically active agents.

If a combination of active agents are administered, then they may be administered simultaneously, separately or sequentially.

The agents may exist as stereoisomers and/or geometric isomers—eg they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The agent for use in the present invention may be administered in the form of a pharmaceutically acceptable salt, especially when the agent is a low molecular weight organic molecule.

Pharmaceutically-acceptable salts are well known to those skilled in the art, and for example include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent for use in the present invention may be readily prepared by mixing together solutions of the agent and the desired acid or base, as appropriate. The salt may precipitate from solution and be collected by filtration or may be recovered by evaporation of the solvent.

The agent for use in the present invention may exist in polymorphic form.

It will be appreciated by those skilled in the art that the agent for use in the present invention may be derived from a prodrug. Examples of prodrugs include entities that have certain protected group(s) and which may not possess pharmacological activity as such, but may, in certain instances, be administered (such as orally or parenterally) and thereafter metabolised in the body to form the agent of the present invention which are pharmacologically active.

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

The present invention also includes the use of zwitterionic forms of the agent for use in the present invention. The terms used in the claims encompass one or more of the forms just mentioned.

The present invention also includes the use of solvate forms of the agent for use in the present invention. The terms used in the claims encompass these forms.

As indicated, the present invention also includes the use of pro-drug forms of the agent for use in the present invention. The terms used in the claims encompass these forms.

As used herein, the term “mimetic” relates to any chemical which includes, but is not limited to, a peptide, polypeptide, antibody or other organic chemical which has the same qualitative activity or effect as a reference agent.

The term “derivative” or “derivatised” as used herein includes chemical modification of an agent. Illustrative of such chemical modifications would be replacement of hydrogen by a halo group, an alkyl group, an acyl group or an amino group.

In one embodiment of the present invention, the agent may be a chemically modified agent.

The chemical modification of an agent of the present invention may either enhance or reduce hydrogen bonding interaction, charge interaction, hydrophobic interaction, Van Der Waals interaction or dipole interaction between the agent and the target.

The present invention also provides a pharmaceutical composition comprising a therapeutically effective amount of the agent(s) for use in the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as or in addition to the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route.

Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets ‘containing excipients such ’ as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, eg as a carrier, diluent or solubiliser. Alpha-, beta- and gamma-cyclodextrins are most commonly used and suitable examples are described in WO-A-91/11172, WO-A-94/02518 and WO-A-98/55148.

If the agent is a protein, then said protein may be prepared iii situ in the subject being treated. In this respect, nucleotide sequences encoding said protein may be delivered by use of non-viral techniques (eg by use of liposomes) and/or viral techniques (eg by use of retroviral vectors) such that the said protein is expressed from said nucleotide sequence.

If the agent is an antisense agent it may be administered using any suitable technique. The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

Antisense oligonucleotides may be administered using methods well known in the art.

The components of the present invention may be administered alone but will generally be administered as a pharmaceutical composition—eg when the components are is in admixture with a suitable pharmaceutical excipient, diluent or carrier selected with regard to the intended route of administration and standard pharmaceutical practice.

For example, the components can be administered (eg orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (eg as a tablet, capsule, or as an ingestable solution), topical, mucosal (eg as a nasal spray or aerosol for inhalation), nasal, parenteral (eg by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

It is particularly preferred if the agent is delivered intravaginally. It is also preferred if the agent is delivered using slow release techniques as are well known in the art.

It is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrastemally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, eg dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, eg using a mixture of ethanol and the propellant as the solvent, which may additionally contain a lubricant, eg sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, diet, mode and time of administration, rate of excretion, drug combination, the severity of the particular condition, and the individual undergoing therapy.

The component(s) of the present invention may be formulated into a pharmaceutical composition, such as by mixing with one or more of a suitable carrier, diluent or excipient, by using techniques that are known in the art.

It is particularly preferred if the agent is adapted to delivery to the female reproductive system. Thus, preferably, the agent is adapted for delivery intravaginally to the cervix. Suitably, the agent is comprised within a pessary for vaginal administration, or is formulated into a gel suitable for administration to the cervix intravaginally. Typically, a vaginal ring may be used for administration.

Other suitable adaptations for delivery to the female reproductive system are readily envisaged by the person skilled in the art. For example, it may be desirable to coat a tampon with the agent. For proteinaceous agents, such as antibodies and variants of CD40 and CD40L, it is preferred if they are contained within liposomes or other delivery vehicles which protect the agent until it reaches the site of administration.

Preferably, the agent is delivered to the female reproductive system via the peripheral blood, for example by using skin patches.

The agent may also be delivered orally. Typically, if the agent is a proteinaceous agent it is delivered in a lipid complex, or is attached to a hydrophobic group, such as a long chain ester.

A further aspect of the invention provides a screening method for predisposition of an individual to a disorder of the female reproductive system, the method comprising obtaining a sample from the individual which contains CD40 and/or CD40L nucleic acid or protein and determining whether CD40 and/or CD40L nucleic acid or protein is present in an amount and/or concentration, or in a form, indicative of such predisposition.

The sample obtained from the individual is any suitable material and typically is a sample (such as a tissue sample) which contains nucleic acid and/or protein. A suitable sample containing both nucleic acid and protein is a nucleated cell from the individual. The cell may be from the individual's reproductive system but it need not be. For example, a white blood cell may be useful in the screening when genetic tests are applied. A tissue sample may be obtained at endometrial biopsy using a Pipelle, Eurosurgical Limited, The Common, Cranleigh, UK.

By “determine whether CD40 and/or CD40L nucleic acid or protein is present in an amount and/or concentration, or form, indicative of such predisposition” we include the meaning determining the amount and/or concentration of CD40 or CD40L protein or mRNA in the sample and comparing its amount and/or concentration to that of a control.

Typically, an investigation may be made of whether and to what extent CD40 is expressed in an endometrial stromal cell of an individual.

The control may be from an individual who is known to be susceptible or from an individual known not to be susceptible. Alternatively, the control may be in the form of a “look-up” data point or table, which records amounts (or concentrations), of protein mRNA which relate to susceptibility or lack of susceptibility.

We also include determining the form of the nucleic acid or protein and determining, by comparison with a control, whether the form is one associated with susceptibility or lack of susceptibility. Although it is possible to measure different forms of protein, it is preferred if the form of DNA is determined (ie polymorphic state). Thus, for example, the method includes determining at any one or more nucleotide positions within the CD40 and/or CD40L gene which base is present and correlating that to susceptibility or lack thereof.

Quantitation of protein or mRNA in a sample may be measured by any suitable method such as ELISA (for proteins) and quantitative reverse transcriptase PCR (RT-PCR) or northern blotting for mRNA.

Determination of the form of a protein may be carried out by, for example, western blotting.

Determination of the form of DNA can be carried out by, for example, DNA sequencing, or by using well known methods such as restriction fragment length polymorphism (RFLP) determination or single nucleotide polymorphism (SNP) analysis.

Control samples can be obtained from suitable sources. Look-up data points or tables can readily be prepared by, for example, correlating the parameters (ie amount and/or concentration of, or form of, nucleic acid or protein) with individuals with the disorder. If necessary, this can be done retrospectively.

The screening method is useful in determining predisposition to any one menstrual dysfunction, pre-term labour or endometriosis; preferably it is used to determine predisposition to endometriosis.

In a particularly preferred embodiment, predisposition to endometriosis is determined by assessing polymorphisms in the CD40 and/or CD40L genes of an individual. Complementary DNAs corresponding to the CD40 and CD40L genes are described in GenBank Accession Nos X60592 and Z15017, respectively.

### EXAMPLES

The present invention will now be described by way of example only in which reference is made to the following Figures:

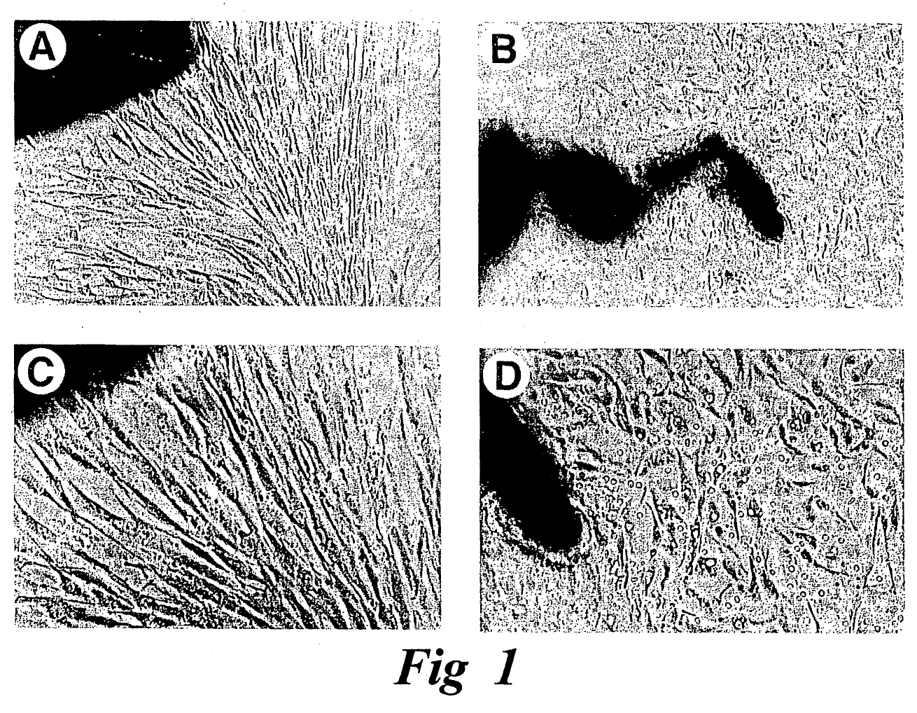

FIG. 1 which presents photographs.

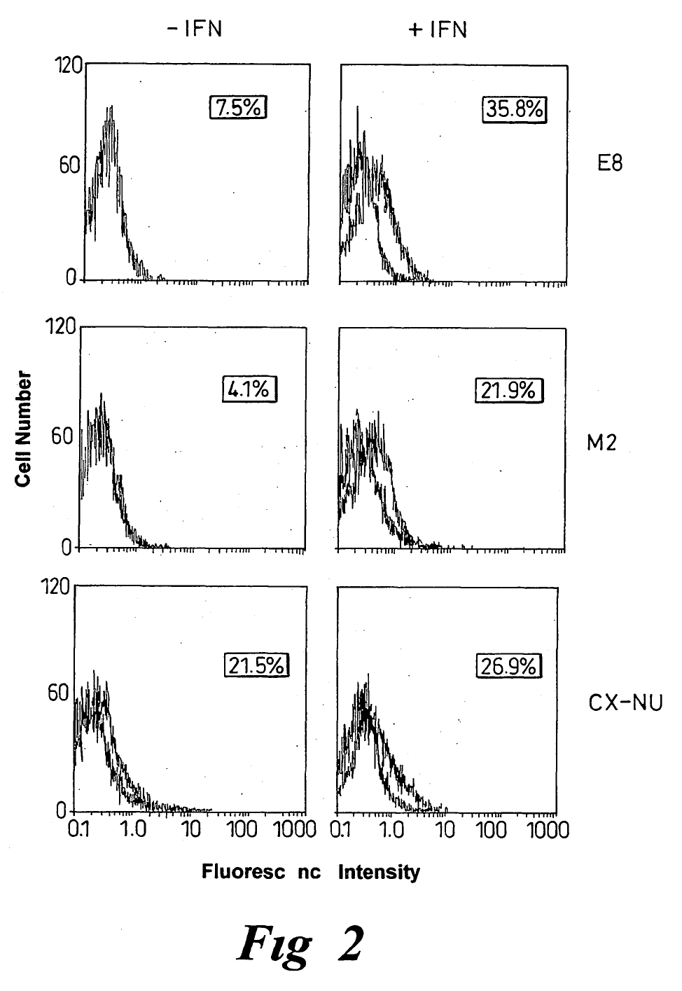

FIG. 2 which presents a graph

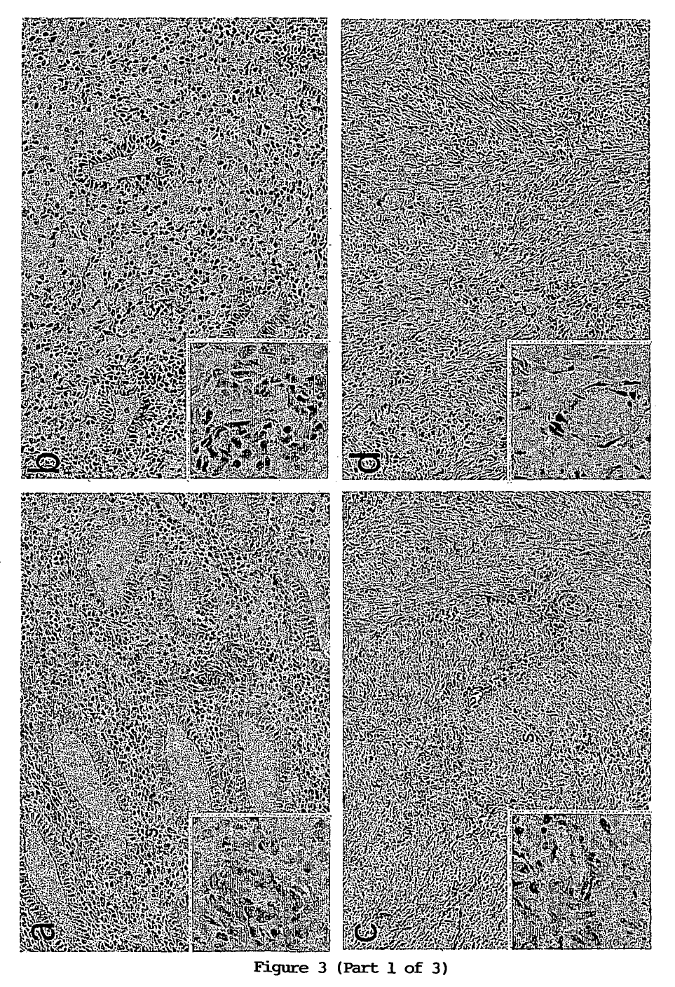

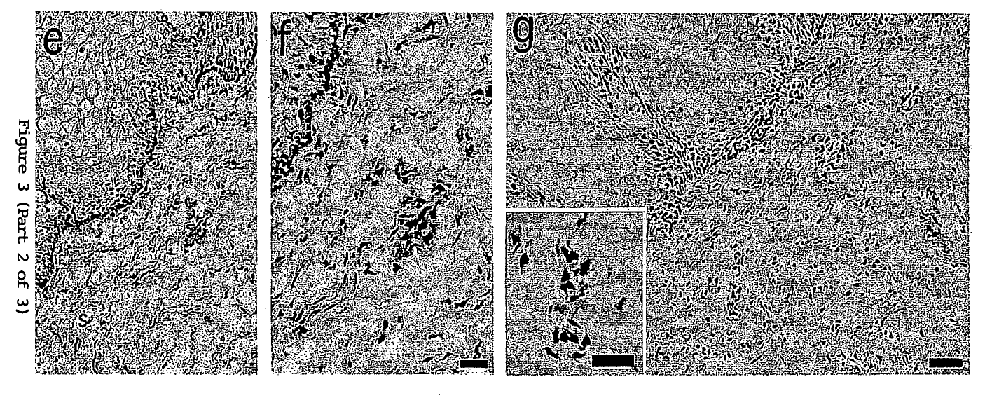

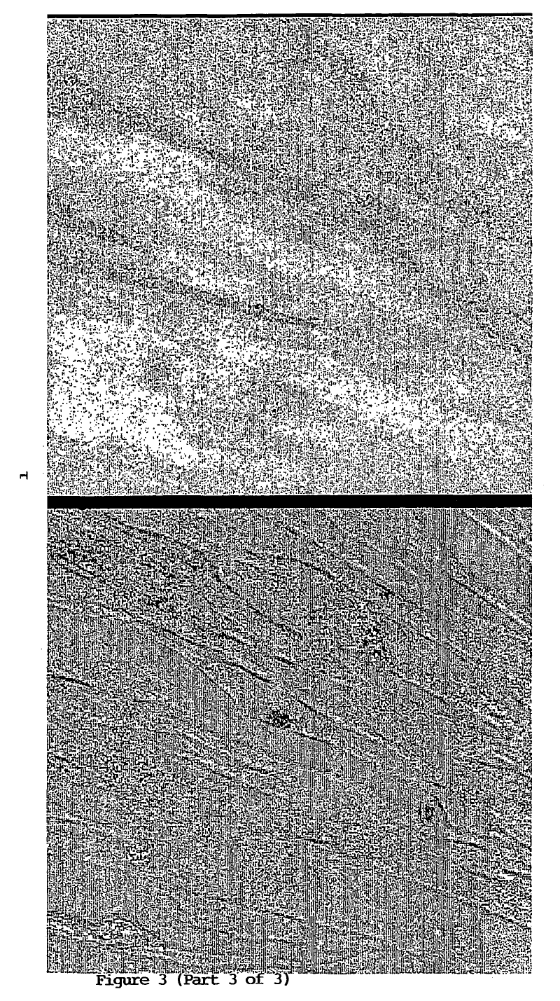

FIG. 3 which presents photographs.

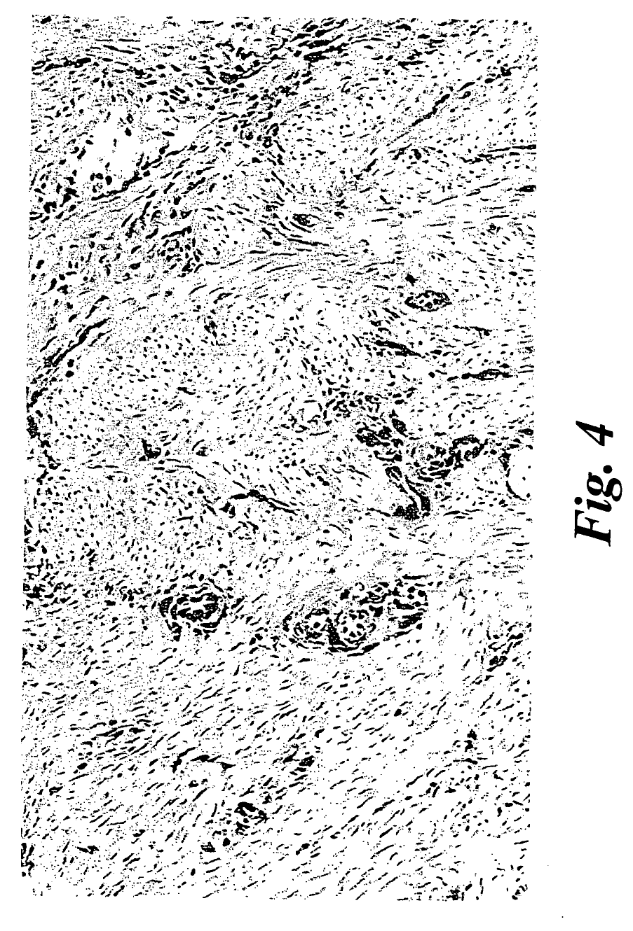

FIG. 4 which presents a photograph.

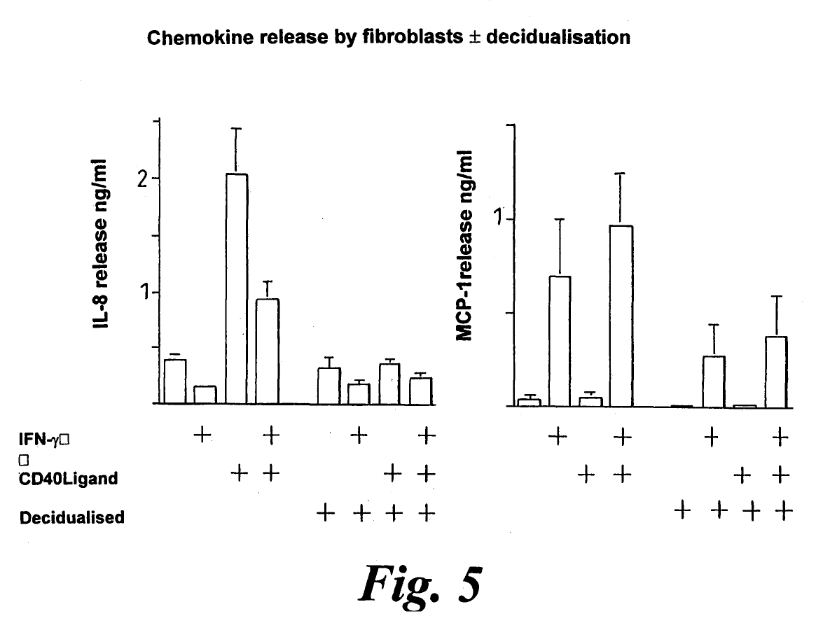

FIG. 5 which presents a graph.

In more detail:

FIG. 1: Outgrowth of fibroblasts from tissue explants (dark areas). (A). Myometrial line 5 (×100). (B). Endometrial line 9 (×100). (C). Myometrial line 5 (×200). (D). Endometrial line 9 (×200).

FIG. 2: Upregulation of fibroblast CD40 expression upon IFNγ treatment. Fibroblasts were incubated with an anti-CD40 monoclonal antibody (light grey histogram) or an equimolar concentration of mouse IgG1 (isotype control; black histogram). Low levels of CD40 were detected on untreated fibroblasts compared to those incubated with mouse IgG1 (see % in boxes). Treatment with IFNγ for 72 hrs increased CD40 expression (see % in boxes). Data are shown for endometrial (E8), myometrial (M2) and cervical (CX-NU) fibroblasts.

FIG. 3: shows immunohistochemical localization of CD40 in human endometrium, myometrium and cervix.

FIG. 4: This photograph shows perivascular distribution of CD40 at endometrial/myometrial border.

FIG. 5: shows chemokine release by fibroblasts±decidualisation.

General Introduction

CD40 is a cell surface receptor initially discovered on cells of the hematopoietic lineage. Its primary role on immune cells is to enhance their activation and hence their production of cytokines and immunomodulatory molecules. Recently, CD40 has also been detected on human fibroblasts. An emerging view of the fibroblast is that it is far more than a structural cell, being capable of intimate interaction with cells of the immune system. In fibroblasts from several tissues the engagement of CD40 with its ligand (CD40L) resulted in secretion of proinflammatory molecules such as IL-6 and IL-8. Currently there is little data regarding the presence of the CD40-CD40L system in female reproductive tissues. This study investigates the expression of CD40 by human endometrium, myometrium and cervix both in situ and in tissue explant-derived fibroblasts. CD40 was detected mainly in the perivascular region of endometrium, myometrium and cervix. Light staining for CD40 was observed in stromal elements. Additionally, the basal epithelium of cervix expressed CD40. Fibroblastic cells derived from all three sources express low levels of. CD40 and this is upregulated with interferon-γ treatment (500U/ml; 72 hr). These data suggest that the CD40-CD40L system may provide a link between the resident structural cells of these reproductive tissues and the infiltrating immune cells or activated platelets which may express CD40L. Henn et al (1998) Nature 391, 591-594 shows expression of CD40L on activated platelets. The possible interaction of CD40 with CD40L may be particularly important during events such as menstruation and cervical ripening where upregulation of the proinflammatory molecules IL-6 and IL-8 is viewed as critical for these processes. In addition, dysregulation of this system may be a contributory factor to problems such as menstrual dysfunction and preterm labour.

Detailed introduction

The normal functioning of the human female reproductive tract is associated with inflammatory-like responses eg menstruation, cervical ripening and parturition (I). These processes are characterised by increased expression of inflammatory mediators, leukocyte infiltration and tissue degradation. It is also likely that inappropriate activation of inflammatory pathways is responsible for pathophysiological situations such as preterm labour and menstrual dysfunction.

The cytokine interleukin-6 (IL-6) and chemokines, interleukin-8 (IL-8; attracts mainly neutrophils) and monocyte chemoattractant peptide 1 (MCP-1; attracts mainly monocytes), are likely to be critical mediators in female reproduction. IL-8 and MCP-1 expression increases in the late secretory phase of the menstrual cycle (2) while the expression of IL-6 becomes apparent in the mid secretory phase and progressively increases in the late secretory and menstrual phase (3). This suggests a role for these mediators in menstruation. In cervix IL-8 production is closely associated with ripening (4) and synergistic actions with PGE2 are reported (5). Parturition also involves upregulated expression of inflammatory mediators and IL-6 in particular has been linked to preterm labour (6). Progesterone is likely to be involved in the control of inflammatory mediator expression in reproductive tissues and a model of progesterone withdrawal has shown increased expression of chemokines in endometrium (7). However, the molecular mechanisms which cause this upregulation remain unclear.

CD40 is a member of the tumour necrosis factor-α (TNFα) receptor family and is expressed on several cell types including B lymphocytes (8), monocytes, vascular endothelial cells (9) and some epithelial cells (10). CD40 ligand (CD40L), the endogenous ligand for CD40, is found on activated T cells, mast cells (11), eosinophils (12) and basophils (11). Interestingly, platelets have an intracellular pool of preformed CD40L which is surface-expressed upon activation (13). The CD40-CD40L system is involved in B cell-T cell signalling events (14) and has also been found to be important in non-haematopoietic cell activation; For example, CD40 has recently been found on human fibroblasts from several sites and interferon-γ (IFNγ) treatment upregulates CD40 expression on these cells (15, 16). Activation of CD40 on some types of fibroblast results in the upregulation of the proinflammatory cytokines IL-6 and IL-8 (16-18):

The CD40-CD40L system has not previously been investigated in normal human endometrium and myometrium although CD40 was recently detected in the squamous epithelium of cervical carcinoma (19). The presence of the CD40-CD40L system in reproductive tissues could provide a key link between the resident structural cells of the tissues and infiltrating immune cells. Such a link would be likely to be involved in the inflammatory-like responses associated with both physiological and pathophysiological reproductive events. This study investigates the expression of CD40 in human endometrium, myometrium and cervix.

Material & Methods

Tissue Collection.

Endometrial (n=24), myometrial (n=26) and non-pregnant cervical biopsies (n=3) were collected from women undergoing gynaecological procedures for benign conditions. All women had regular menstrual cycles (25-35 days) and had not received any form of hormonal treatment in the 3 months preceding biopsy. Endometrial biopsies were collected throughout the menstrual cycle (proliferative, n=10; early-mid secretory, n=8; late secretory, n=3). Histological dating according to the criteria of Noyes et al (20) and circulating progesterone levels were consistent with the date of the last menstrual period. Cervical biopsies were collected from the anterior lip region of the cervix. Cervical biopsies from pregnant women (n=8) were collected from nulliparous patients during the first trimester of pregnancy. [Written informed consent was received from all patients prior to biopsy collection. Ethical approval was received from Lothian Research Ethics Committee and the Ethics Committee of the Karolinska hospital.]

Immunohistochemistry.

Frozen tissue sections were lightly fixed in neutral buffered formalin (NBF) for 10 min at room temperature. Endogenous peroxidase activity was blocked with 3% hydrogen peroxide (BDH Laboratory Supplies, Poole, UK) in distilled water for 10 min at room temperature. Diluted normal horse serum (Vectastain 4002; Vector Laboratories, Peterborough, UK) was applied to all tissue sections for 20 min in a humidified chamber at room temperature. Tissue sections were incubated overnight at 4° C. with 50 μl of mouse anti-CD40 antibody (G28-5, diluted in horse serum (21, 22)). The primary antibody was substituted with an equimolar concentration of mouse immunoglobulin (Vector Laboratories) in negative control sections. Sections were incubated with biotinylated horse-anti mouse immunoglobulin (Vector Laboratories) and then an avidin-biotin peroxidase detection system (both for 60 min at room temperature; Elite ABC 6101; Vector Laboratories). The peroxidase substrate diaminobenzidine (DAB, Vector Laboratories) was used to identify positive staining. Sections were counterstained with Harris's haemotoxylin (Pioneer Research Chemicals Ltd, Colchester, UK), dehydrated in ascending grades of alcohol and mounted from xylene in Pertex (Cellpath plc, Hemel Hempstead, UK). Results are shown in FIG. 4.

Derivation of Fibroblast Strains.

Endometrial and myometrial explants were cultured in complete media, ie RPMI 1640 (Sigma, Poole, Dorset, UK) supplemented with 10% fetal calf serum (FCS; Mycoplex, PAA Laboratories, Teddington, UK), penicillin (50 μg/ml; Sigma), streptomycin (50 μg/ml; Sigma) and gentamycin (5 μg/ml; Sigma). Cervical fibroblasts were established in MEM supplemented with 10% donor calf serum and then cultured as above. Tissue was cut up into pieces approximately 1 mm3. Explants were then placed under glass coverslips in 100 mm diameter tissue culture dishes (Corning Costar, High Wycombe, UK) resulting in the outgrowth of fibroblasts from individual tissues as previously described (15, 18). On some occasions tissues were also digested in dispase II (2.4U/ml; Boehringer Mannheim, Lewes, UK) for 45 min at 37° C. and the resultant cell suspensions were cultured in 25 cm2 culture flasks (Corning Costar). Both techniques yielded fibroblast strains (endometrial n—3; myometrial n=5; cervical n=3) and no differences were observed in terms of efficiency of generation of fibroblast strains. Fibroblast strains were named E (endometrial), M (myometrial) or CX (cervical) to describe the tissue of origin and were subsequently numbered or lettered for research tracking purposes. Immunostaining showed the cells to be vimentin positive but CD45 and cytokeratin negative. This is consistent with a fibroblast phenotype. The cells also expressed the fibroblast marker Thy-1. All cells used in experiments were from as early passage as possible.

Flow Cytometry and Immunofluorescence.

Fibroblasts were cultured with or without human interferon-γ (500U/ml; Peprotech, London, UK) for 72 hr at 37° C. The cells were lightly trypsinised, washed and then resuspended in PBS supplemented with 0.1% azide and 1% bovine serum albumin (BSA). Rapid treatment with trypsin does not appreciably cleave CD40 from these cells (17). The cells (1×10⁶) were then incubated with anti-human CD40 (G28-5) for 30 min on ice. Mouse IgG or mouse IgG1 (Sigma) were used as negative controls. The cells were washed and then incubated with fluorescein-conjugated (FITC) goat-anti mouse Ig (1:100 dilution; Cappel, ICN Biomedical, Costa Mesa, Calif.) for 30 min on ice. After washing the cells were resuspended in PBS+0.1% azide+1% BSA. Analysis was on a Coulter Model XL flow cytometer. Data analysis and subtraction were completed using an Immuno-4 programme (Coulter Electronics, Hialeah, Fla.).

CD40 expression on fibroblasts was also examined in situ by immunofluorescence. Cells were cultured on 8 well chamber slides and then sequentially stained with anti-human CD40 and FITC goat-anti-mouse Ig as detailed above. Fibroblasts were examined by standard immunofluorescence on an Olympus Provis System and by a Zeiss LSM 510 laser scanning microscope.

RESULTS

Localisation of CD40 in Human Endometrium, Myometrium and Cervix.

Immunohistochemistry showed positive CD40 immunoreactivity in the perivascular region of all endometrial biopsies studied (n=21). The area around the blood vessels that expressed CD40 was several cell layers deep. These cells have been termed myofibroblasts (25). Moderate immunostaining was also present in some fibroblast-like cells particularly in the basal and subglandular regions of the stroma (FIG. 3a). Very faint epithelial staining and evidence of white blood cell CD40 expression were observed in some biopsies. No significant differences in CD40 staining were observed during the menstrual cycle.

In myometrium (n=21) positive immunoreactivity was observed in the perivascular region with faint immunostaining also apparent in some stromal fibroblasts (FIG. 3c).

Immunostaining of cervical biopsies (n=S) also showed CD40 to be localised around blood vessels and in some stromal cells. Additionally, in contrast to endometrium, strong positive immunoreactivity was present in the basal epithelium. Surface epithelium did not express CD40 (FIG. 3e). This is similar to observations in human skin where both dermal fibroblasts and the basal epithelium express CD40 (10).

FIG. 3 shows immunohistochemical localization of CD40 in human endometrium, myometrium and cervix. All insets show blood vessels at higher magnification. Arrows show the location of blood vessels. Nuclei were counterstained with haematoxylin. Scale bar in main pictures=50 μm; insets=201 μm. (A). Early secretory endometrium (progesterone level: 39.71 nmol/L). Immunoreactivity is present in the perivascular area and in some stromal cells (s). Only very faint immunostaining is found in the glandular epithelium. (B). Endometrium. Negative control. Primary antibody replaced with mouse immunoglobulin at equimolar concentrations. (C). Myometrium. CD40 is present in the perivascular cells. Faint immunostaining is also found in some stromal cells (s). (D). Myometrium. Negative control. (E). Cervix (day 65 of gestation). CD40 immunoreactivity is found in the perivascular area, some stromal cells (s) and in the basal epithelium. No CD40 is present in surface epithelium. (F). Cervix. Basal epithelium and blood vessels shown at higher magnification. (G). Cervix. Negative control. (H & I). CD40 staining on an endometrial fibroblast strain treated with IFNγ. The E8 strain of endometrial fibroblast was treated with or without IFNγ for 72 hours. The cells were then stained with an isotype control antibody or with the G28-5 anti-CD40 antibody followed by a FITC-labelled anti-goat anti-mouse Ig. The images also show a phase contrast view of the cells. These images show the surface staining pattern of CD40. The non-IFNγ treated cells show weak staining compared to the IFNγ treated fibroblasts (data not shown). These results were reflective of the flow cytometry data in FIG. 2.

Expression of CD40 on Endometrial, Myometrial and Cervical Fibroblasts.

Human fibroblast lines were derived from endometrial (n=3), myometrial (n=5) and cervical (n=3) explants. The resultant cells (FIG. 1) were morphologically consistent with a fibroblast phenotype and expressed the fibroblast markers Thy-1 and vimentin (data not shown). These cells also failed to display the epithelial marker cytokeratin and CD45 a marker of bone marrow derived cells. They did not morphologically resemble endothelial cells. All cells displayed the characteristics detailed above indicating that the cell populations were pure. Surface CD40 expression was measured by flow cytometry and immunofluorescence. Flow cytometric analysis revealed that all lines, with the exception of myometrial fibroblast strain 9, displayed a small amount of CD40. No consistent differences were observed in CD40 expression from fibroblast strains derived from biopsies taken at various times during the menstrual cycle. FIG. 2 shows results for endometrial 8 (E8), myometrial 2 (M2) and cervical NU (CX-NU) fibroblast lines. Approximately 8% of the untreated endometrial fibroblasts expressed CD40 while 4% of the myometrial fibroblasts displayed the molecule. Twenty two % of the cervical fibroblasts expressed CD40. These figures rose to approximately 36%, 22% and 27%, respectively, after treatment with IFNγ for 72 hrs. In general the entire peaks shifted towards increased CD40 fluorescence which suggested that the majority of the fibroblasts were upregulating their display of CD40. Further evidence for this was obtained using in situ immunofluorescence in concert with conventional and confocal microscopy. Compared with non-IFNγ treated cells, most of the IFNγ-treated fibroblasts showed upregulation of CD40 with a surface punctate pattern of expression (FIGS. 3h and i).

Chemokine Release by Fibroblasts±Decidualisation.

Five separate endometrial stomal cell preparations were grown in 10% fetal calf serum in RPMI medium enriched with fibroblast growth factor and medroxy progesterone acetate (MPA) at 1 Molar concentration. 5×10⁵ cells were cultured per well and cells were decidualised for 5 days with 8-bromo cyclic AMP 5×10⁴ Molar and MPA (1×10⁻⁶ M). Cells with medium changed at two days of culture. Cells treated with Interferon 7 (IFNγ) were cultured with 20 ng/ml IFNγ for 48 hours and then rested by growth in 10% FCS in RPMI 1640 for 24 hours. Treatment with CD40 ligand 20 ng/ml and enhancer antibody (10 ng/ml) (both from Alexis, Nottingham) was carried out for 24 hours. Cells were washed with saline and harvested with Tri reagent (Sigma Poole, Dorset) and RNA was extracted according to manufacturers instructions. RNA was reverse-transcribed to cDNA and quantitative PCR was used to confirm decidualisation and CD40 expression. Medium was harvested and IL-8 and MCP-1 were measured by ELISA as previously reported (Denison, Kelly et al (1997) J. Repro. Immunol. 34, 225-240; Denison, Grant et al (1998) Mol. Human Repro. 5, 220-226). Data are shown as means±standard errors of the mean (N=5). Results are given in FIG. 5.

Discussion

An emerging paradigm is that fibroblasts, rather than serving only as inert structural cells, are key elements for interacting with and controlling cells of the immune system. This is accomplished as fibroblasts express surface markers that allow interaction with hematopoietic cells and also secrete chemokines controlling immune cell traffic. Previously, CD40 was detected on human fibroblasts from tissues such as lung. Engagement of fibroblast CD40 served as a potent “activation” step, stimulating synthesis of IL-6 and IL-8 (15, 17, 18). We report here for the first time that fibroblasts from human endometrium, myometrium and cervix also express CD40. Engagement by CD40L “activates” these fibroblasts inducing synthesis of cytokines such as IL-8. These exciting findings indicate that the CD40-CD40L system is likely to provide a universal mechanism for regulation of proinflammatory cytokine expression in uterine fibroblasts. It is also possible, but as yet unknown, that fibroblasts from reproductive tissues may express a unique spectrum of cytokines distinct from fibroblasts derived from other tissues.

The detection of CD40 in situ and on freshly derived fibroblast strains in female reproductive tissues is particularly interesting as inflammatory mechanisms (with increased chemokine release and infiltration of immune cells) are key elements in reproductive physiology. We propose that the presence of CD40 allows interaction of resident fibroblasts with immune cells. Conventionally, the CD40-CD40L interaction is regarded as occurring via cellular contact. However, our data does not exclude the possibility that, in the reproductive tract, the interaction is via soluble CD40L or CD40L on the surface of platelets (thrombocytes). In endometrium, expression of IL-8 and MCP-1 increases in the perivascular region prior to menstruation (2). Upregulation of IL-8 mRNA and protein is also observed in a model of progesterone withdrawal (7). The detection of CD40 by immunohistochemistry in the perivascular region suggests a mechanism allowing upregulation of these mediators. Although CD40 expression remains constant throughout the menstrual cycle it is possible that the presence of CD40L in endometrium increases at the time of menstruation possibly by increased extravasation of platelets. This would allow increased signalling via the CD40-CD40L system. Additionally, COX-2 expression increases perivascularly in the late secretory phase of the menstrual cycle (2) and after progesterone withdrawal (7) and this may influence vascular permeability (see Colditz et al (1990) Immunol. Cell Biol. 68, 397-403). CD40 activation may also be responsible for this as increased COX-2 and PGE2 expression occur upon stimulation of lung fibroblasts (31). The expression of CD40 by perivascular cells also suggests that the CD40-CD40L system may contribute to the tissue remodelling that occurs during menstruation. The cells of the endometrial perivascular area (pre-decidual stromal cells) are likely to be of myofibroblast lineage as they have been shown to express a smooth muscle actin (25). Myofibroblasts have contractile activity and are thought to be involved in wound healing. Another interesting potential role for the CD40-CD40L system is in cervical ripening and parturition. IL-8 release increases in the cervix at the time of ripening (4, 32) and in the lower segment myometrium during labour (30).

The activation of CD40 μl vivo relies on the presence of CD40L-expressing cells or thrombocytes at the local site. Several cell types present in reproductive tissues may express CD40L. In endometrium there are resident T cells, macrophages and mast cells throughout the menstrual cycle with the appearance of eosinophils premenstrually and uterine NK cells in the mid-late secretory phase (34, 35). Myometrium is infiltrated by T cells and macrophages at the onset of labour. Mast cells are present at low levels throughout pregnancy with higher levels in the non-pregnant state (30). In the ripening cervix mast cells and eosinophils (36) are present and macrophages are present from late pregnancy onwards (37). Activated platelets may also be present in reproductive tissues at times of infection, bleeding or increased vascular permeability.

In addition to a seminal role in physiological reproductive events there are pathophysiological problems that may also involve the CD40-CD40L system. Dysfunctional menstrual bleeding is likely to involve aberrant expression of uterine mediators. For instance those associated with the perivascular region of endometrium and myometrium may contribute to menorrhagia and it seems likely that dysregulation of the CD40 system could be involved. The importance of the CD40 system in menstrual pathology will depend on the identification of the relevant source of the ligand. Further studies are necessary to determine whether T cells or platelets can activate the system in endometrium. However, it is clear that the extravasation of blood at the initiation of menstruation will deliver large numbers of platelets close to the site of CD40 expression. These platelets may express CD40L allowing activation of CD40 (13). The resultant cytokine expression could modulate vessel permeability and thus act as a negative feedback regulator.

Premature labour is associated with increased expression of proinflammatory cytokines in the uterine cavity particularly during infection (6, 38). The CD40-CD40L system may be activated during uterine infection and therefore be involved in the upregulation of cytokines. Recently, CD40 has been detected in cervical carcinoma with CD40L present on infiltrating T cells. CD40-CD40L interactions were suggested as a possible regulator of chemokine expression in carcinoma cells indicating that the CD40-CD40L system may be involved in reproductive cancers (19).

Currently, disruption of the CD40-CD40L system using biologics has proven extremely successful in blunting transplant rejection (39), pulmonary injury (40, 41) and autoimmune disease (42). Disruption of the CD40-CD40L system in the female reproductive tract may also prove valuable in treating reproductive disorders.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### CD40-CD40L REFERENCES

1. Asimakopoulos, F. A.; White, N. J.; Nacheva, E. P.; Green, A. R.: The human CD40 gene lies within chromosome 20q deletions associated with myeloid malignancies. Brit. J Haemat. 92: 127-130, 1996.

2. Clark, E. A.: CD40: A cytokine receptor in search of a ligand. Tissue Antigens 35: 33-36, 1990.

3. Kato, K.; Cantwell, M. J.; Sharma, S.; Kipps, T. J.: Gene transfer of CD40-ligand induces autologous immune recognition of chronic lymphocytic leukemia B cells. J Clin. Invest. 101: 1133-1141, 1998.

4. Lafage-Pochitaloff, M.; Herman, P.; Birg, F.; Galizzi, J.-P.; Simonetti, J.; Mannoni, P.; Banchereau, J.: Localization of the human CD40 gene to chromosome 20, bands ql2-ql3.2. Leukemia 8: 1172-1175, 1994.

5. Mach, F.; Schonbeck, U.; Sukhova, G. K.; Atkinson, E.; Libby, P.: Reduction of atherosclerosis in mice by inhibition of CD40 signalling. Nature 394: 200-203, 1998.

6. Stamenkovic, I.; Clark, E. A.; Seed, B.: A B-lymphocyte activation molecule related to the nerve growth factor receptor and induced by cytokines in carcinomas. EMBO J. 8: 1403-1410, 1989.

7. Tan, J.; Town, T.; Paris, D.; Mori, T.; Suo, Z.; Crawford, F.; Mattson, M. P.; Flavell, R. A.; ullan, M. Microglial activation resulting from CD40-CD40L interaction after beta-amyloid stimulation. Science 286: 2352-2355, 1999.

### EXAMPLE REFERENCES

1. Kelly R W. 1994 Pregnancy maintenance and parturition: the role of prostaglandin in manipulating the immune and inflammatory response. Endocr Rev. 15: 684-706.

2. Jones R L, Kelly R W, Critchley H O D. 1997 Chemokine and cyclooxygenase-2 expression in human endometrium coincides with leukocyte accumulation. Hum Reprod. 12: 1300-1306.

3. Tabibzadeh S, Kong Q F, Babaknia A, May L T. 1995 Progressive rise in the expression of interleukin-6 in human endometrium during menstrual cycle is initiated during the implantation window. Hum Reprod. 10: 2793-2799.

4. Sennstrom M K, Brauner A, Lu Y, Granstrom L M, Malmstrom A L, Ekman G E. 1997 Interleukin-8 is a mediator of the final cervical ripening in humans. Eur J Obstet Gynecol Reprod Biol. 74: 89-92.

5. Colditz I G. 1990 Effect of exogenous prostaglandin E2 and actinomycin D on plasma leakage induced by neutrophil-activating peptide-interleukin-8. Immunol Cell Biol. 68: 397-403.

6. Romero R. Avila C, Santhanam U, Sehgal P B. 1990 Amniotic fluid interleukin 6 in preterm labor. Association with infection. J Clin Invest. 85: 1392-1400.

7. Critchley H O D, Jones R L, Lea R G, et al. 1999 Role of inflammatory mediators in human endometrium during progesterone withdrawal and early pregnancy. J Clin Endocrinol Metab. 84: 240-248.

8. Stamenkovic I, Clark E A, Seed B. 1989 A B-lymphocyte activation molecule related to the nerve growth factor receptor and induced by cytokines in carcinomas. Embo J. 8: 1403-1410.

9. Hollenbaugh D, Mischel-Petty N, Edwards C P, et al. 1995 Expression of functional CD40 by vascular endothelial cells. J Exp Med. 182: 33-40.

10. Gaspari A A, Sempowski G D, Chess P, Gish J, Phipps R P. 1996 Human epidermal keratinocytes are induced to secrete interleukin-6 and co-stimulate T lymphocyte proliferation by a CD40-dependent mechanism. Eur J Immunol. 26: 1371-1377.

11. Gauchat J F, Henchoz S, Mazzei G, et al. 1993 Induction of human IgE synthesis in B cells by mast cells and basophils. Nature 365: 340-343.

12. Gauchat J F, Henchoz S, Fattah D, et al. 1995 CD40 ligand is functionally expressed on human eosinophils. Eur J Immunol. 25: 863-865.

13. Henn V, Slupsky J R, Grafe M, et al. 1998 CD40 ligand on activated platelets triggers an inflammatory reaction of endothelial cells. Nature 391: 591-594.

14. Clark L B, Foy T M, Noelle R J. 1996 CD40 and its ligand. Adv Immunol. 63: 43-78.

15. Fries K M, Sempowski G D, Gaspari A A, Blieden T., Looney R J, Phipps R P. 1995 CD40 expression by human fibroblasts. Clin Immunol Immunopathol. 77: 42-51.

16. Sempowski G D, Rozenblit J, Smith T J, Phipps R P. 1998 Human orbital fibroblasts are activated through CD40 to induce proinflammatory cytokine production. Am J Physiol. 274: C707-714.

17. Sempowski G D, Chess P R, Phipps R P. 1997 CD40 is a functional activation antigen and B7-independent T cell costimulatory molecule on normal human lung fibroblasts. J Immunol. 158: 4670-4677.

18. Sempowski G D, Chess P R, Moretti A J, Padilla J, Phipps R P, Blieden T M. 1997 CD40 mediated activation of gingival and periodontal ligament fibroblasts. J Periodontol. 68: 284-292.

19. Altenburg A, Baldus S E, Smola H, Pfister H, Hess S. 1999 CD40 ligand-CD40 interaction induces chemokines in cervical carcinoma cells in synergism with IFN-gamma. J Immunol. 162:4140-4147.

20. Noyes R W, Hertig A T, Rock J. 1950 Dating the Endometrial Biopsy. Fertility and Sterility 1: 3-25.

21. Clark E A, Ledbetter J A. 1986 Activation of human B cells mediated through two distinct cell surface differentiation antigens, Bp35 and Bp50. Proc Natl Acad Sci USA 83: 4494-4498.

22. Clark E A, Yip T C, Ledbetter J A, et al. 1988 CDw4O and BLCa-specific monoclonal antibodies detect two distinct molecules which transmit progression signals to human B lymphocytes. Eur J Immunol. 18: 451-457.

24. Ida N, Sakurai S, Kawano G. 1994 Detection of monocyte chemotactic and activating factor (MCAF) in normal blood and urine using a sensitive ELISA. Cytokine 6: 32-39.

25. Oliver C, Montes M J, Galindo J A, Ruiz C, Olivares E G. 1999 Human decidual stromal cells express alpha-smooth muscle actin and show ultrastructural similarities with myofibroblasts. Hum Reprod. 14:1599-1605.

30. Thomson A J, Telfer J F, Young A, et al. 1999 Leukocytes infiltrate the myometrium during human parturition: further evidence that labour is an inflammatory process [In Process Citation]. Hum Reprod. 14: 229-236.

31. Zhang Y, Cao H J, Graf B, Meekins H, Smith T J, Phipps R P. 1998 CD40 engagement up-regulates cyclooxygenase-2 expression and prostaglandin E2 production in human lung fibroblasts. J Immunol. 160: 1053-1057.

32. Kelly R W, Leask R, Calder A A. 1992 Choriodecidual production of interleukin-8 and mechanism of parturition. Lancet 339: 776-777.

34. Jeziorska M, Salamonsen L A, Woolley D E. 1995 Mast cell and eosinophil distribution and activation in human endometrium throughout the menstrual cycle. Biol Reprod. 53: 312-320.

35. Loke Y W, King A. Uterine Mucosal Lymphocytes. In: Y. W. Loke and A. King, eds. Uterine Mucosal Lymphocytes. Cambridge: Cambridge University Press; 102-129.

36. Knudsen U B, Uldbjerg N, Rechberger T, Fredens K. 1997 Eosinophils in human cervical ripening. Eur J Obstet Gynecol Reprod Biol. 72: 165-168.

37. Bokstrom H, Brannstrom M, Alexandersson M, Norstrom A. 1997 Leukocyte subpopulations in the human uterine cervical stroma at early and term pregnancy. Hum Reprod. 12: 586-590.

38. Romero R, Ceska M, Avila C, Mazor M, Behnke E, Lindley I. 1991 Neutrophil attractant/activating peptide-1/interleukin-8 in term and preterm parturition. Am J Obstet Gynecol. 165: 813-820.

39. Kirk A D, Burkly L C, Batty D S, et al. 1999 Treatment with humanized monoclonal antibody against CD154 prevents acute renal allograft rejection in nonhuman primates [see comments]. Nat Med. 5: 686-693.

40. Adawi A, Zhang Y, Baggs R, Finkelstein J, Phipps R P. 1998 Disruption of the CD40-CD40 ligand system prevents an oxygen-induced respiratory distress syndrome. Am J Pathol. 152: 651-657.

41. Adawi A, Zhang Y, Baggs R, et al. 1998 Blockade of CD40-CD40 ligand interactions protects against radiation-induced pulmonary inflammation and fibrosis. Clin Immunopathol. 89: 222-230.

42. Kalled S L, Cutler A H, Datta S K, Thomas D W. 1998 Anti-CD40 ligand antibody treatment of SNF1 mice with established nephritis: preservation of kidney function. J Immunol. 160: 2158-2165.

1. A method of treating a disorder of the female reproductive system in an individual the method comprising modulating the CD40-CD40L system in the said reproductive system.

2. A method according to claim 1 wherein the disorder is not a reproductive cancer.

3. A method according to claim 1 wherein the disorder is a benign condition.

4. A method according to claim 1 wherein the disorder is one involving the uterine vasculature.

5. A method according to claim 1 to 3 wherein the disorder is any one of menstrual dysfunction, pre-term labour, endometriosis and fibroids.

6. A method of treating a disorder of the female reproductive system in an individual the method comprising disrupting the CD40-CD40L system in the said reproductive system.

7. A method according to any one of claims 1 to 5 wherein the individual is administered an agent which modulates the CD40-CD40L system.

8. A method according to claims 6 and 7 wherein the agent disrupts the CD40-CD40L system.

9. A method according to claim 8 wherein the agent targets CD40 or CD40L.

10. A method according to claim 7 or 8 wherein the agent is a biologic.

11. A method according to any one of claims 7 to 10 wherein the agent is one or more of an antibody, a CD40L variant, a soluble CD40 or an anti-CD40 or anti-CD40L antisense nucleic acid.

12. A method according to any one of claims 1 to 9 wherein the agent is a CD40L antagonist.

13. A method according to any one of claims 7 to 12 wherein the agent is an antibody.

14. A method according to any one of claims 7 to 12 wherein the agent is an antisense nucleic acid.

15. A method according to any one of claims 1 to 14 wherein the individual is a human female.

16. Use of an agent which modulates the CD40-CD40L system in the manufacture of a medicament for treating a disorder of the female reproductive system.

17. Use according to claim 16 wherein the agent disrupts the CD40-CD40L system.

18. A therapeutic system for treating a disorder of the female reproductive system the system comprising an agent which modulates the CD40-CD40L system in the said reproductive system.

19. A therapeutic system according to claim 18 wherein the agent disrupts the CD40-CD40L system.

20. A therapeutic system according to claim 18 or 19 further comprising any of an oral contraceptive pill (combined and progestogen only), progestogens and antigestagens.

21. An agent which modulates the CD40-CD40L system for use in treating a disorder of the female reproductive system in an individual.

22. An agent according to claim 21 wherein the agent disrupts the CD40-CD40L system.

23. Use of an agent which modulates the CD40-CD40L system for treating a disorder of the female reproductive system in an individual.

24. Use of an agent according to claim 23 wherein the agent disrupts the CD40-CD40L system.

25. A pharmaceutical preparation adapted for delivery to the female reproductive system comprising an agent which modulates the CD40-CD40L system.

26. A pharmaceutical preparation according to claim 25 wherein the agent disrupts the CD40-CD40L system.

27. A screening method for predisposition of an individual to a disorder of the female reproductive system, the method comprising obtaining a sample from the individual which contains CD40 and/or CD40L nucleic acid or protein and determining whether CD40 and/or CD40L nucleic acid or protein is present in an amount and/or concentration, or in a form, indicative of such predisposition.

28. A screening method, according to claim 27 wherein the disorder of the female reproductive system is any one of a menstrual dysfunction, preterm labour or endometriosis.

29. A screening method according to claim 27 wherein the disorder of the female reproductive system is endometriosis.

30. A screening method according to any one of claims 27 to 29 wherein a polymorphism in the CD40 and/or CD40L gene is determined and correlated to the said predisposition.
